(19) United States
(12) Plant Patent Application Publication
(10) Pub. No.: US 2019/0183023 P1
(43) Pub. Date: Jun. 13, 2019

(54) Chrysanthemum plant named 'DOMLICOBLA'

(50) ;

(71) Applicant: DUMMEN GROUP B.V., DE LIER (NL)

(72) Inventor: Peter Wain, Locks Heath (GB)

(73) Assignee: DUMMEN GROUP B.V., DE LIER (NL)

(21) Appl. No.: 16/350,604

(22) Filed: Dec. 7, 2018

Related U.S. Application Data
(60) Provisional application No. 62/708,405, filed on Dec. 8, 2017.

(51) Int. Cl. A01H 6/14

(52) U.S. Cl. PLT/294

(57) ABSTRACT

A new and distinct cultivar of Chrysanthemum plant named ‘Domlicobla’, characterized by its upright plant habit; moderately vigorous growth habit; large anemone type inflorescences with white-colored ray florets; response time about 50 days under controlled photoperiodic treatments; and good postproduction longevity.

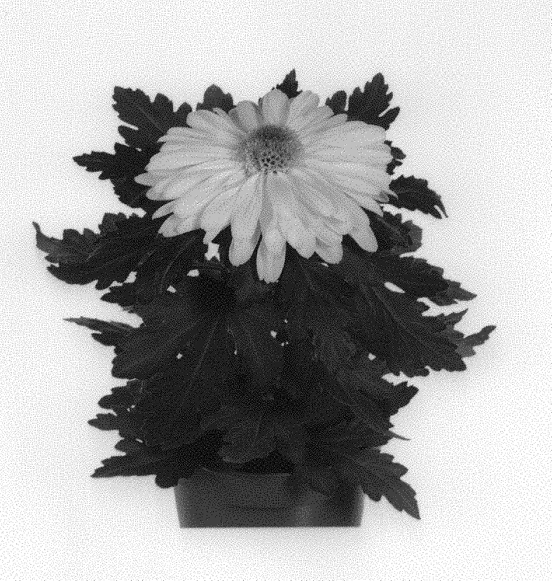

Botanical designation: Chrysanthemum X morifolium

Cultivar denomination: ‘DOMLICOBLA’.

### BACKGROUND OF THE INVENTION

The present invention relates to a new and distinct Chrysanthemum plant, botanically known as Chrysanthemum X morifolium, commercially grown as a potted Chrysanthemum plant, referred to as code number 65860 in U.S. Provisional Patent Application Ser. No. 62/708,405 and hereinafter referred to by the name ‘Domlicobla’.

The new Chrysanthemum plant is a product of a planned breeding program conducted by the Inventor in Fareham, Hampshire, United Kingdom. The objective of the breeding program is to create new potted Chrysanthemum plants with numerous attractive inflorescences.

The new Chrysanthemum plant originated from a cross-pollination made in January, 2014 by the Inventor in Fareham, Hampshire, United Kingdom of a proprietary selection of Chrysanthemum X morifolium identified as code number 807880, not patented, as the female, or seed, parent with a proprietary selection of Chrysanthemum X morifolium identified as code number 807040, not patented, as the male, or pollen, parent. The new Chrysanthemum plant was discovered and selected by the Inventor as a single flowering plant from within the progeny of the stated cross-pollination in a controlled greenhouse environment in Fareham, Hampshire, United Kingdom in September, 2014.

Asexual reproduction of the new Chrysanthemum plant by terminal vegetative cuttings was first conducted in Fareham, Hampshire, United Kingdom in December, 2014. Asexual reproduction by terminal vegetative cuttings has shown that the unique features of this new Chrysanthemum plant are stable and reproduced true to type in successive generations.

### SUMMARY OF THE INVENTION

Plants of the new Chrysanthemum have not been observed under all possible combinations of environmental conditions and cultural practices. The phenotype may vary somewhat with variations in environmental conditions such as temperature, daylength and light intensity, without, however, any variance in genotype.

The following traits have been repeatedly observed and are determined to be the unique characteristics of ‘Domlicobla’. These characteristics in combination distinguish ‘Domlicobla’ as a new and distinct Chrysanthemum plant:

1. Upright plant habit.

2. Moderately vigorous growth habit.

3. Large anemone type inflorescences with white-colored ray florets.

4. Response time about 50 days under controlled photoperiodic treatments.

5. Good postproduction longevity.

Plants of the new Chrysanthemum can be compared to plants of the female parent selection. Plants of the new Chrysanthemum differ primarily from plants of the female parent selection in inflorescence type as plants of the new Chrysanthemum have anemone type inflorescences whereas plants of the female parent selection have semi-decorative type inflorescences.

Plants of the new Chrysanthemum can be compared to plants of the male parent selection. Plants of the new Chrysanthemum differ primarily from plants of the male parent selection in inflorescence form as plants of the new Chrysanthemum have anemone type inflorescences whereas plants of the male parent selection have single type. inflorescences.

Plants of the new Chrysanthemum can be compared to plants of Chrysanthemum X morifolium ‘Fichrycoswhi’, disclosed in U.S. Plant patent application Ser. No. 15/732,509. In side-by-side comparisons, plants of the new Chrysanthemum differ primarily from plants of ‘Fichrycoswhi’ in the following characteristics:

1. Plants of the new Chrysanthemum have anemone type inflorescences whereas plants of ‘Fchrycoswhi’ have decorative type inflorescences.

2. Ray florets of plants of the new Chrysanthemum are mostly flat whereas ray florets of plants of ‘Fichrycoswhi’ are incurved.

### BRIEF DESCRIPTION OF THE PHOTOGRAPH

The accompanying photograph illustrates the overall appearance of the new Chrysanthemum plant showing the colors as true as it is reasonably possible to obtain in colored reproductions of this type. Colors in the photograph may differ slightly from the color values cited in the detailed botanical description which accurately describe the colors of the new Chrysanthemum plant.

The photograph is a side perspective view of a typical flowering plant of ‘Domlicobla’ grown in a 14-cm container.

### DETAILED BOTANICAL DESCRIPTION

The aforementioned photograph and following observations and measurements describe plants grown during the spring in 14-cm containers in a glass-covered greenhouse in Fareham, Hampshire, United Kingdom and under cultural practices typical of commercial garden Chrysanthemum production. During the production of the plants, day and night temperatures ranged from 17° C. to 21° C. and light levels averaged 6,000 lux. Plants were grown under long day/short night conditions for about two weeks and then grown under short day/long night conditions to induce inflorescence initiation and development. Plants were ten weeks old when the photograph and detailed description were taken. In the following description, color references are made to The Royal Horticultural Society Colour Chart, Fifth Edition, except where general terms of ordinary dictionary significance are used.
Botanical classification: Chrysanthemum X morifolium ‘Domlicobla’.
Parentage:
   Female, or seed, parent.—Proprietary selection of Chrysanthemum X morifolium identified as code number 807880, not patented.
   Male, or pollen, parent.—Proprietary selection of Chrysanthemum X morifolium identified as code number 807040, not patented.
Propagation:
   Type.—Terminal vegetative cuttings.
   Time to initiate roots, summer.—About ten days at temperatures about 21° C.
   Time to initiate roots, winter.—About twelve days at temperatures about 21° C.
   Time to produce a rooted young plant, summer.—About three weeks at temperatures about 21° C.
   Time to produce a rooted young plant, winter.—About four weeks at temperatures about 21° C.
   Root description.—Fine, fibrous; typically light brown in color, actual color of the roots is dependent on substrate composition, water quality, fertilizer type and formulation, substrate temperature and physiological age of roots.
   Rooting habit.—Freely branching; dense.
Plant description:
   Plant and growth habit.—Herbaceous semi-decorative type potted Chrysanthemum; grown as a single-stem plant, stems upright; moderately vigorous growth habit; medium growth rate.
   Plant height.—About 12 cm.
   Plant width.—About 30 cm.
   Internode length.—About 8 mm.
   Stem strength.—Strong.
   Texture.—Fine pubescence.
   Color.—Close to 138B.
Leaf description:
   Arrangement.—Alternate, simple.
   Length.—About 8 cm.
   Width.—About 5.5 cm.
   Shape.—Palmately-lobed; roughly ovate with three to five lobes.
   Apex.—Acuminate.
   Base.—Attenuate.
   Margin.—Slightly dentate and palmately lobed; sinuses between lateral lobes mostly divergent.
   Texture, upper surface.—Fine pubescence; slightly rough.
   Texture, lower surface.—Fine pubescence; waxy; veins prominent.
   Color.—Developing leaves, upper surface: Close to 139A. Developing leaves, lower surface: Close to 137B. Fully expanded leaves, upper surface: Close to N137A; venation, close to 138B. Fully expanded leaves, lower surface: Close to 137B; venation, close to 138B.
   Petioles.—Length: About 1.9 cm. Diameter: About 2 mm. Texture, upper surface: Fine pubescence; slightly rough. Texture, lower surface: Fine pubescence; waxy. Color, upper and lower surfaces: Close to 138B.
Inflorescence description:
   Form and flowering habit.—Anemone type inflorescence form with ligulate-shaped ray florets; inflorescences borne on terminals above and beyond the foliar plane; disc and ray florets arranged acropetally on a capitulum; grown as a single stem disbud type only one inflorescence per plant is allowed to develop during the flowering season.
   Fragrance.—Mildly fragrant; pungent, herbaceous.
   Flowering response.—Plants flower uniformly about 50 days after starting short day/long night photoperiodic treatments.
   Inflorescence longevity.—Good postproduction longevity; inflorescences maintain good color and substance for about two to three weeks on the plant; inflorescences persistent.
   Inflorescence buds.—Height: About 7 mm. Diameter: About 8 mm. Shape: Oblate. Color: Close to 143A.
   Inflorescence diameter.—Large, about 12.5 cm.
   Inflorescence height.—About 2.5 cm.
   Disc diameter.—About 3 cm.
   Receptacles.—Height: About 4 mm. Diameter: About 7 mm. Shape: Oblate. Color: Close to 144D.
   Ray florets.—Number of ray florets per inflorescence: About 97 arranged in about four whorls. Orientation: Initially upright, then about 90° from vertical; slightly concave. Length: About 4.5 cm. Width: About 1 cm. Shape: Ligulate. Apex: Emarginate. Base: Fused into a short tube. Margin: Entire. Texture, upper and lower surfaces: Smooth, glabrous; double-keeled. Color: When opening, upper and lower surfaces: Close to NN155D. Fully opened, upper and lower surfaces: Close to NN155D; color does not change with development.
   Disc florets.—Number of disc florets per inflorescence: About 196 massed at the center of the receptacle. Length: About 1.9 cm. Diameter: About 2 mm. Shape: Tubular, elongated; apices, acute. Texture, inner and outer surfaces: Smooth, glabrous. Color, when opening: Apex: Close to 154A. Mid-section: Close to 14C. Base: Close to 150D. Color, fully opened: Apex: Close to NN155D. Mid-section: Close to 150C. Base: Close to 150B.
   Phyllaries.—Number ofphyllaries per inflorescence: About 23 arranged in about two whorls. Length: About 1 cm. Width: About 4 mm. Shape: Lanceolate. Apex: Acute. Base: Obtuse. Margin: Entire. Texture, upper surface: Smooth, glabrous; waxy. Texture, lower surface: Fine pubescence; waxy. Color, upper and lower surfaces: Close to 143A.
   Reproductive organs.—Androecium: None observed. Gynoecium: Present only on ray florets. Pistil length: About 7 mm. Stigma shape: Bi-parted. Stigma color: Close to 5A. Style length: About 0.5 mm. Style color: Close to 150C. Ovary color: Close to 150D.
   Seeds and fruits.—To date, seed and fruit production has not been observed on plants of the new Chrysanthemum.
Pathogen & pest resistance: Resistance to pathogens and pests common to Chrysanthemum plants has not been observed on plants of the new Chrysanthemum to date.
Garden performance: Plants of the new Chrysanthemum have demonstrated good garden performance and to tolerate temperatures from about 0° C. to about 35° C.

It is claimed:

1. A new and distinct Chrysanthemum plant named ‘Domlicobla’ as illustrated and described.
